(19) United States
(12) Patent Application Publication
(10) Pub. No.: US 2023/0372378 A1
(43) Pub. Date: Nov. 23, 2023

(54) COMPOSITIONS AND METHODS FOR ALLEVIATING CONDITIONS CAUSED BY ABNORMAL SUBCUTANEOUS DEPOSIT OF ADIPOSE TISSUE OR FAT

(71) Applicant: Caliway Biopharmaceuticals Co., Ltd., New Taipei City (TW)

(72) Inventor: Yu-Fang LING, New Taipei City (TW)

(21) Appl. No.: 18/317,564

(22) Filed: May 15, 2023

Related U.S. Application Data
(60) Provisional application No. 63/342,284, filed on May 16, 2022.

(51) Int. Cl. A61K 31/7048; A61K 31/352; A61K 31/05; A61K 31/12; A61P 35/00

(52) U.S. Cl. CPC A61K 31/7048; A61K 31/352; A61K 31/05; A61K 31/12; A61P 35/00

(57) ABSTRACT

Methods for alleviating a condition caused by abnormal subcutaneous deposit of adipose tissue or fat in a subject in need thereof an effective amount of a composition comprising a first active agent, which comprises (i) a resveratrol compound, a curcumin compound, a quercetin compound, a puerarin compound, or a combination thereof, and (ii) a pharmaceutically acceptable carrier.

### CROSS REFERENCE TO RELATED APPLICATIONS

This application claims the benefit of the filing dates of U.S. Provisional Application No. 63/342,284, filed May 16, 2022, the entire contents of which are incorporated by reference herein.

### FIELD OF THE INVENTION

The present disclosure relates to pharmaceutical compositions and their use for alleviation in alleviating (e.g., treating) conditions caused by abnormal subcutaneous deposit of adipose tissues or fat. More specifically, the present disclosure relates to compositions comprising micelles formed by lipophilic and/or hydrophilic therapeutic agents and suitable surfactants for alleviating (e.g., treating) conditions caused by abnormal subcutaneous deposit of adipose tissues or fat.

### BACKGROUND OF THE INVENTION

Lipomas are defined as a common subcutaneous tumor composed of adipose cells, often encapsulated by a thin layer of fibrous tissue. Clinically, lipomas can present at any place in a body may even cause death if they grow in vital regions. Lipomas are associated with various medical conditions, including Dercum's disease (Adiposis dolorosa). Dercum's disease is a rare disorder characterized by chronic pain due to multiple lipomas in the adipose tissue. Kucharz et al., Reumatologia 57(5):281-287 (2019).

Dercum's disease involves long-term chronic and relapsing lipoma. No specific treatment exists for Dercum's disease. Treatment is directed toward the specific symptoms that are apparent in each individual and is primarily focus on easing the characteristic painful episodes. Patients will have multiple painful lipomas or fat masses with abnormal accumulation of subcutaneous fat on the trunk and limbs, accompanied by spontaneous severe pain in the area, and it lasts more than 3 months. The above severe chronic pain will make the patient unable to live a normal life. In addition, the symptoms will not spontaneously relieve or disappear, painful lipomas may become larger in a short time, even after surgical removal or liposuction, new lipomas or fat lumps will still grow. The cause of the disease has not yet been determined, and it is likely to be related to adipose tissue metabolic disorders.

According to statistics, there are currently about 124,500 Dercum's disease patients in the United States, and about 240,000 patients in Europe. The incidence rate of women is about 5 to 30 times more than men. At present, there is no drug or product approved for marketing in the world to treat Dercum's disease, and only surgery or other drugs can relieve symptoms. Liposuction or surgical excision are relatively common treatment methods, but they have obvious side effects and high risks, and the postoperative recovery period is longer. And even with surgical removal or liposuction, new lipomas or fat masses will grow again.

Nowadays various painkillers (analgesics) have been tried with limited effectiveness. Injections of corticosteroids have also been used to treat individuals with Dercum's disease. However, in one reported case in the medical literature, the use of high-doses of corticosteroids was linked as a possible cause of the disease. Intravenous administration of the pain reliever lidocaine and/or ketamine may provide temporary relief from pain in some people.

Surgical excision of lipomas may temporarily relieve symptoms although the generation of inflammation (part of the healing process) during and after the surgery may induce more lipomas to develop in that area. Liposuction has been used as a supportive treatment for some individuals with Dercum's disease and may provide an initial reduction in pain and improvement in quality of life. These effects may lessen over time.

It is therefore of great importance to develop effective and safer therapies with wound-free care, lower infection risk or minimized sequelae for alleviating lipoma and treating diseases associated with lipoma.

### SUMMARY OF THE INVENTION

The present disclosure is based, at least in part, on the development of pharmaceutical compositions comprising suitable therapeutic agents and surfactant, which form micelles, for use in alleviating (e.g., treating) conditions caused by abnormal subcutaneous deposit of adipose tissues or fat, e.g., lipoma, liposarcoma, lipedema, and other such conditions as known in the art or provided herein. For example, exemplary compositions provided herein successfully reduced lipoma sizes and alleviated lipoma-associated pain. See Examples below.

Accordingly, in one aspect, the present disclosure features a method for alleviating a condition caused by abnormal subcutaneous deposit of adipose tissue or fat in a subject. The method comprises administering to a subject in need of the treatment an effective amount of a composition containing (a) an active agent (e.g., a resveratrol compound, a curcumin compound, a quercetin compound, a puerarin compound, or a combination thereof); and (b) a pharmaceutically acceptable carrier. In some instances, the resveratrol compound can be resveratrol. In other instances, the curcumin compound can be curcumin. Alternatively or in addition, the quercetin compound can be quercetin, and/or the puerarin compound can be puerarin.

The pharmaceutically acceptable carrier can be a pharmaceutically acceptable non-ionic surfactant (e.g., Tween 80, polyoxyl 15 hydroxystearate (solutol HS 15), a polyoxyethylene castor oil derivative, or a combination thereof). Some preferred non-ionic surfactants have a hydrophilic-lipophilic balance value (HLB value) greater than 10. In some embodiments, the weight ratio of the active agent to the non-ionic surfactant can be 1:5-1:500. The composition of the active agent and non-ionic surfactant may be in the form of micelles.

In other embodiments, the composition may further comprise additional micelles formed by a non-ionic surfactant (e.g., as described above) and one or more hydrophilic therapeutic agent (e.g., green tea extract, epicatechin, epicatechingallate, epigallocatechin, gallocatechingallate, gallocatechin, catechingallate, catechin, epigallocatechin gallate (EGCG), caffeine, carnitine, L-carnitine, synephrine, chlorogenic acid, and other hydrophilic drugs, or a combination thereof). The weight ratio of the hydrophilic therapeutic agent to the non-ionic surfactant in the second micelles may also be 1:5-1:500. Additionally, the weight ratio of the first active agent to the hydrophilic therapeutic agent may be 30:1 to 1:10.

Any of the micelles contained in the composition disclosed herein (e.g., those comprising the active agent as disclosed herein) may have a diameter less than 50 nm (e.g., about 1 to about 50 nm, or about 10-25 nm) and/or a polydispersity index (PDI) value less than 0.4.

In some examples, the composition may be formulated for parenteral administration, for example, for injection, implantation or transdermal administration. The composition formulated for injection can be in the form of powder (e.g., lyophilized powder), sterilized suspension, injectable solution, injectable emulsion, or intravenous fluid. In some examples, the composition maybe placed in a microneedle device for injection. Alternatively, the composition may be formulated for transdermal administration, e.g., in a form of ointment, lotion, liniment, cream, gel, dressing, emulsion, film, patch, poultice, cataplasm, topical powder, or topical solution.

In some embodiments, any of the methods disclosed herein may be performed by administering any of the compositions disclosed herein via a parenteral route (e.g., topical administration or local injection). In some examples, the composition may be administered to a local site, e.g., at or near the lipoma or liposarcoma. In some instances, the local site may be at the thigh, buttock, lower limb, pelvic region, or abdomen, where lipomas or liposarcomas frequently occur. The composition may be administered to a subject once or multiple times depending on the size of the lipoma or liposarcoma for treatment.

In some embodiments, the condition for treatment by any of the methods disclosed herein may be a tumor of fat tissue. In some examples, the tumor of fat tissue may be a benign tumor, for example, lipoma. In some instances, the lipoma can be a nodular lipoma. In other instances, the lipoma may be a diffuse lipoma. In other examples, the tumor of fat tissue may be a malignant tumor, for example, liposarcoma, Proteus syndrome, PTEN hamartoma tumor syndrome, Gardner syndrome, Familial multiple lipomatosis, Cowden syndrome, Madelung's disease, painful lipomas, angiolipomas, lipomatosis, subcutaneous adipose tissue diseases, lipedema, a rare adipose disorder (RAD), or soft tissue sarcoma.

In specific examples, the method disclosed herein is for treating a disease involving lipomas. Such a disease can be the Dercum's disease. In other examples, the method disclosed herein is for treating liposarcoma. In yet another example, the method disclosed herein is for treating lipedema.

Also provided herein are pharmaceutical compositions as disclosed herein for use in alleviating conditions caused by abnormal subcutaneous deposit of adipose tissues and/or fat (e.g., lipoma or diseases associated with lipoma such as Dercum's disease; or liposarcoma). Further, the present disclosure provides uses of any of the pharmaceutical compositions as disclosed herein for manufacturing a medicament for use in treating any of the target conditions as disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram illustrating comparative pain scale chart.

FIGS. 2A-2B include diagrams showing the effect in reducing lipoma size. FIG. 2A: average lipoma size. FIG. 2B: average changes in lipoma size.

FIGS. 3A-3B include diagrams showing the effect in pain relieving. FIG. 3A: average comparative pain scale score. FIG. 3B: average change of comparative pain scale score.

FIGS. 4A-4B include diagrams showing cell viability (%) (FIG. 4A) and cytotoxicity of liposarcoma (%) (FIG. 4B) of SW872 cells treated by the compositions having curcumin at concentrations.

### DETAILED DESCRIPTION OF THE INVENTION

Disclosed herein are pharmaceutical compositions and therapeutic uses thereof for alleviating (e.g., treating) conditions caused by abnormal subcutaneous deposit of adipose tissue or fat, including the diseases and disorders provided herein. The pharmaceutical compositions disclosed herein may comprise one or more active agents, which may be a resveratrol compound, a curcumin compound, a quercetin compound, a puerarin compound, or a combination thereof. The one or more active agents may form micelles (a first plurality of micelles) with a suitable pharmaceutically acceptable non-ionic surfactant, e.g., as those disclosed herein. In some embodiments, the pharmaceutical composition may further comprise a second plurality of micelles, which can be formed by a suitable pharmaceutically acceptable non-ionic surfactant and a hydrophilic agent as disclosed herein. Also provided herein are methods for alleviating (e.g., treating) conditions caused by abnormal subcutaneous deposit of adipose tissues or fat using any of the pharmaceutical compositions disclosed herein, e.g., lipoma or liposarcoma.

### I. Pharmaceutical Compositions

The pharmaceutical compositions disclosed herein comprise one or more of a resveratrol compound, a curcumin compound, a quercetin compound, and a puerarin compound as the active agent for alleviating conditions caused by abnormal subcutaneous deposit of adipose tissue or fat, e.g., lipoma or liposarcoma. The active agent as disclosed herein may form micelles with suitable non-ionic surfactants such as those disclosed herein (the first plurality of micelles). The pharmaceutical composition disclosed herein may further comprise a second plurality of micelles formed by a hydrophilic agent and a suitable non-ionic surfactant.

### (A) Active Agents

Exemplary active agents provided herein include resveratrol compounds, curcumin compounds, quercetin compounds, and puerarin compounds.

A resveratrol compound may have the structure of Formula (I) or a salt thereof:

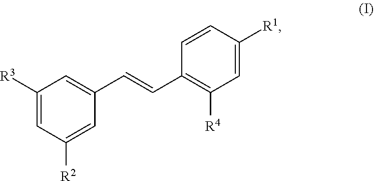

in which each of R¹, R², R³ and R⁴ is independently H, halo (e.g., F, Cl, or Br), hydroxyl, alkyl, alkenyl, alkynyl, alkoxy, thiol, and amine. In some embodiments, a resveratrol compound may have the structure of Formula (Ia) or a salt thereof:

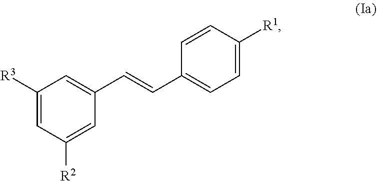

in which each of R¹, R², and R³ is as defined above.

In one example, the resveratrol compound is resveratrol (with all of R¹-R³ being —OH, R⁴ being —H). Alternatively, a resveratrol compound may have a suitable substitution at one or more suitable positions in resveratrol as known to those skilled in the art.

A curcumin compound as disclosed herein may have the structure of Formula (II) or a salt thereof:

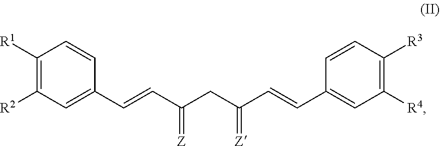

in which each of R¹, R², R³, and R⁴ is independently H, halo (e.g., F, Cl, or Br), hydroxyl, alkyl, alkenyl, alkynyl, alkoxy, thiol, and amine; Z is CH₂, NH, O, or S; and Z′ is CH₂, NH, O, or S. In some embodiments, a curcumin compound is a curcuminoid, for example, curcumin, demethoxycurcumin, or bisdemethoxycurcumin. In specific examples, the curcumin compound is curcumin. Alternatively, a curcumin compound may have a suitable substitution at one or more suitable positions in curcumin as known to those skilled in the art.

In some embodiments, a curcumin compound may have the structure of Formula (IIa) or a salt thereof:

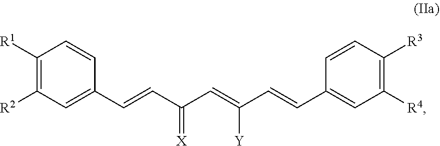

in which each of R¹, R², R³, and R⁴ is independently H, halo (e.g., F, Cl, or Br), hydroxyl, alkyl, alkenyl, alkynyl, alkoxy, thiol, and amine; X is CH₂, NH, O, or S; and Y is CH₃, NH₂, OH, or SH. In some embodiments, a curcumin compound is a curcuminoid, for example, curcumin, demethoxycurcumin, or bisdemethoxycurcumin. In specific examples, the curcumin compound is curcumin. Alternatively, a curcumin compound may have a suitable substitution at one or more suitable positions in curcumin as known to those skilled in the art.

A quercetin compound as disclosed herein may have the structure of Formula (III) or a salt thereof:

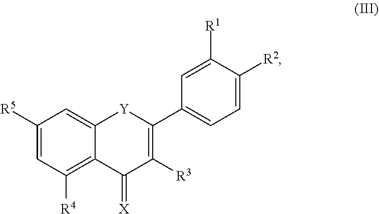

in which each of R¹, R², R³, R⁴, and R⁵ is independently H, halo (e.g., F, Cl, or Br), hydroxyl, alkyl, alkenyl, alkynyl, alkoxy, thiol, and amine; and X and Y are each independently CH₂, NH, O, or S. In specific examples, the quercetin compound is quercetin. Alternatively, a quercetin compound may have a suitable substitution at one or more suitable positions in quercetin as known to those skilled in the art.

A puerarin compound as disclosed herein may be a compound of Formula (IV) or a salt thereof:

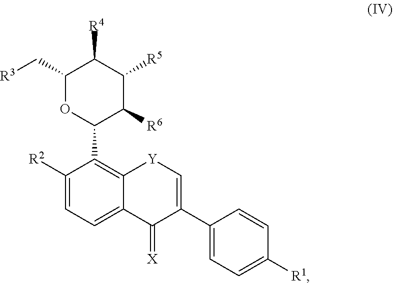

in which each of R¹, R², R³, R⁴, R⁵, and R6 is independently H, halo (e.g., F, Cl, or Br), hydroxyl, alkyl, alkenyl, alkynyl, alkoxy, thiol, and amine; and X and Y are each independently CH₂, NH, O, or S. In some examples, the puerarin compound is puerarin. Alternatively, a purerarin compound may have a suitable substitution at one or more suitable positions in puerarin as known to those skilled in the art.

“Alkyl” refers to a linear, saturated, acyclic, monovalent hydrocarbon radical or branched, saturated, acyclic, monovalent hydrocarbon radical, having from one to three carbon atoms attached to the rest of the molecule by a single bond, e.g., methyl, ethyl, n-propyl, or 1-methylethyl (iso-propyl). An optionally substituted alkyl radical is an alkyl radical that is optionally substituted, valence permitting, by one, two, three, four, or five substituents independently selected from the group consisting of halo, cyano, nitro, oxo, hydroxyl, thio, or amino.

“Alkenyl” refers to a linear, acyclic, monovalent hydrocarbon radical or branched, acyclic, monovalent hydrocarbon radical, containing a carbon-carbon double bond, having two or three carbon atoms attached to the rest of the molecule by a single bond, e.g., ethenyl, or propenyl. An optionally substituted alkenyl radical is an alkenyl radical that is optionally substituted, valence permitting, by one, two, or three substituents independently selected from the group consisting of: halo, cyano, nitro, hydroxyl, thio, or amino.

“Alkynyl” refers to a linear, acyclic, monovalent hydrocarbon radical or branched, acyclic, monovalent hydrocarbon radical containing a triple bond and having two or three carbon atoms attached to the rest of the molecule by a single bond, e.g., ethynyl, or propynyl. An optionally substituted alkynyl radical is an alkynyl radical that is optionally substituted by one substituent selected from the group consisting of: halo, cyano, nitro, hydroxyl, thio, or amino.

“Alkoxy” refers to a radical of the formula —ORa where Ra is a hydrogen or an alkyl radical as defined above containing one to three carbon atoms. The alkyl part of the optionally substituted alkoxy radical is optionally substituted as defined above for an alkyl radical.

“Amino” refers to a radical of the formula —NRbRc where Rb and Rc are each hydrogen, or an alkyl radical as defined above containing one to three carbon atoms. The alkyl part of the optionally substituted amino radical is optionally substituted as defined above for an alkyl radical.

“Thiol” refers to a radical of the formula —SRd where Rd is a hydrogen or an alkyl radical as defined above containing one to three carbon atoms. The alkyl part of the optionally substituted thiol radical is optionally substituted as defined above for an alkyl radical.

“Hydrophilic” as used herein refers to a molecule or molecules that have a hydrophilic-lipophilic balance of greater than 10, e.g., using the Griffin method.

### (B) Hydrophilic Therapeutic Agents

In some embodiments, the pharmaceutical compositions disclosed herein may further comprise one or more hydrophilic therapeutic agents as described herein, which may benefit treatment of the adipose tissue/fat deposit caused conditions, as compared with a pharmaceutical composition comprising only the active agent disclosed herein. The hydrophilic therapeutic agents may be effective in alleviating the conditions as disclosed herein. Alternatively or in addition, the hydrophilic therapeutic agent may enhance the efficacy of the active agent, and/or reduce side effects associated with the active agent.

Hydrophilic therapeutic agents are agents that are soluble in water. Exemplary hydrophilic therapeutic agents include, but are not limited to, green tea extract, flavonoids, including those based on catechin (e.g., catechin, catechin gallate), gallocatechin (e.g., gallocatechin, gallocatechin gallate), epicatechin (e.g., epicatechin, epicatechin gallate), epigallocatechin (e.g., epigallocatechin, epigallocatechin gallate), xanthines, including methylxanthines (e.g., caffeine), carnitine, L-carnitine, synephrine, and chlorogenic acid. Combinations of the foregoing agents may also qualify as the hydrophilic therapeutic agents used herein. Alternatively, other hydrophilic therapeutic agents may be combined with the present technology to aid in the treatment of lipomas. In some embodiments, the hydrophilic therapeutic agent may be combined with a second non-ionic surfactant, preferably forming micelles.

In some embodiments, the weight ratio of the active agent to the hydrophilic therapeutic agent is between 30:1 to 1:10, e.g., between 30:1 to 1:5, between 30:1 to 1:1, between 30:1 to 5:1, between 30:1 to 10:1, between 30:1 to 20:1, between 20:1 to 1:10, between 10:1 to 1:10, between 5:1 to 1:10, between 1:1 to 1:10, between 1:5 to 1:10, between 20:1 to 1:5, between 10:1 to 1:1, or between 5:1 to 1:1.

### (C) Non-Ionic Surfactants

The pharmaceutical composition disclosed herein may comprise one or more non-ionic surfactants, which may form micelles with the active agent and/or the hydrophilic therapeutic agent as disclosed herein. Non-ionic surfactants used in the present technology are beneficial at least in the formation of micelles in the composition. In some embodiments, micelles have the effect of encapsulating the active agent and/or the hydrophilic therapeutic agent.

Non-ionic surfactants used in any of the pharmaceutical compositions disclosed herein preferably have a hydrophilic-lipophilic balance (HLB) value greater than 10. The non-ionic surfactant is used in the present technology in a ratio as set forth above. Exemplary non-ionic surfactants include, but are not limited to, polysorbate 80 (Tween® 80), polyoxyl 15 hydroxystearate (Solutol® HS-15), polyoxyethylene castor oil derivatives (e.g., polyoxyl 35 castor oil (Kolliphor® ELP), polyoxyl 40 hydrogenated castor oil (Koliphor® RH40) and polyoxyl 60 hydrogenated castor oil (Cremophor® RH60), Polyoxyethylene (12) glyceryl laurate (UNIGLY ML-212), Polyoxyl 20 Stearate (Myrj™ S20), Polyoxyl 40 Stearate (Myrj™ S40), Polyoxyl 12 Cetostearyl Ether (Kolliphor® CS 12) and Polyoxyl 20 Cetostearyl Ether (Kolliphor® CS 20).

In some instances, the non-ionic surfactant may be a polyoxyethylene derivative, also known as Pegylated excipient. In some examples, the polyoxyethylene derivative is a PEG castor oil derivative, which are materials obtained by reacting varying amounts of ethylene oxide with either castor oil or hydrogenated castor oil. Examples include PEG-35 castor oil and PEG-40 hydrogenerated Castor Oil. In other examples, the polyoxyethylene derivative is a PEG ester, which can be manufactured by reacting a polyethylene glycol with a fatty acid. Examples include PEG-40 Stearate and PEG-15 Hydroxystearate. In some instances, the ester may be a sorbitan fatty acid ester (e.g., PEG-20 sorbitan monooleate, PEG-40 sorbitan monooleate, PEG-60 sorbitan monooleate, PEG-80 sorbitan monooleate, PEG-20 sorbitan isostearate, PEG-30 sorbitan tetraoleate, PEG-40, -60 sorbitan tetraoleate, PEG-40 sorbitan diisostearate, or PEG-60 sorbitan tetrastearate), an alkyl glyceride (e.g., PEG-8 Caprylic/capric glycerides, PEG-32 hydrogenated palm glycerides, or PEG-32 Lauroyl glycerides), or an alkyl ether (e.g., PEG-6 cetostearyl ether, PEG-12 cetostearyl ether, PEG-20 cetostearyl ether, PEG-10 cetyl ether, PEG-20 cetyl ether, PEG-4 lauryl ether, PEG-23 lauryl ether, PEG-2 oleyl ether, PEG-10 oleyl ether, PEG-20 oleyl ether, PEG-2 stearyl ether, PEG-10 stearyl ether, PEG-21 stearyl ether, or PEG-100 stearyl ether). In yet other instances, the ester may be a laurate (e.g., PEG-2 laurate, PEG-4 laurate, PEG-6 laurate, PEG-8 laurate. PEG-9-14 laurate, PEG-20 laurate, PEG-32-150 laurate, or PEG-12 glyceryl laurate), a dilaurate (e.g., PEG-2 dilaurate, PEG-4 dilaurate, or PEG-6-150 dilaurate), or a stearate (e.g., PEG-2 stearate, PEG-3 stearate, PEG-4 stearate, PEG-4 isostearate, PEG-5-7 stearate, PEG-6-8 isostearate, PEG-8 stearate, PEG-9 stearate, PEG-10 stearate, PEG-10-isostearate, PEG-12 isostearate, PEG-12-18 stearate, PEG-20 stearate, PEG-23-45 stearate, PEG-40 stearate, PEG-50 stearate, PEG-100 stearate, PEG-75-150 stearate, or PEG-6 and PEG-32 palmitostearate), a glyceryl stearate (e.g., Glyceryl stearate/PEG-40 stearate, Glyceryl stearate/PEG-100 stearate, PEG-120 glyceryl stearate, PEG-20 methyl glucose sesquistearate, or PEG-25 propylene glycol stearate), a distearate (e.g., PEG-2 distearate, PEG-3-120 distearate, PEG-150 distearate, or PEG-175 distearate), a hydroxystarate (e.g., PEG-15 hydroxystearates).

In some instances, the non-ionic surfactant may be a castor oil derivative (e.g., PEG-35 castor oil or PEG-40 castor oil) or a hydrogenated castor oil (e.g., PEG-40 hydrogenated castor oil, PEG-54 hydrogenated castor oil, or PEG-60 hydrogenated castor oil).

Additional examples of non-ionic surfactants include PEG-15 cocamine, Vitamin E polyethylene glycol succinate, PEG-75 lanolin, and PEG-120 methyl glucose dioleate.

### (D) Micelles and Preparations Thereof

Any of the pharmaceutical compositions may comprise micelles formed by a first non-ionic surfactant as disclosed herein and one or more active agents, such as a resveratrol compound (e.g., resveratrol), a curcumin compound (e.g., a curcumin), a quercetin compound (e.g., a quercetin), a puerarin compound (e.g., a puerarin), or a combination thereof. In case the pharmaceutical composition further comprises one or more hydrophilic therapeutic agents as disclosed herein, the pharmaceutical composition may further comprise a second non-ionic surfactant, which form micelles with the hydrophilic therapeutic agents. In that case, the pharmaceutical composition may comprise a first plurality of micelles formed by the first non-ionic surfactant and the active agent and a second plurality of micelles formed by the second non-ionic surfactant and the hydrophilic therapeutic agent. In some examples, the first and second non-ionic surfactant are identical. In other examples, the first non-ionic surfactant is different from the second non-ionic surfactant.

In some embodiments, the micelles may comprise the non-ionic surfactant and the active agent at a suitable weight ratio, which lead to suitable particle sizes and/or suitable polydispersity index (PDI) values. For example, a suitable weight ratio between the active agent and the non-ionic surfactant may range from about 1:5-1:500, for example, between about 1:5-1:8, between about 1:5-1:10, between about 1:5-1:20, between about 1:5-1:40, between about 1:5-1:100, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, or between 1:150-1:500. It was reported that micelles formed at the suitable weight ratio between the active agent as disclosed herein and the non-ionic surfactant would lead to suitable particle sizes (e.g., less than 50 nm) and suitable PDI values (e.g., <0.4). See U.S. Pat. No. 10,610,496, the relevant disclosures of which are incorporated by reference for the subject matter and purpose referenced herein.

In some embodiments, the pharmaceutical compositions may further comprise micelles formed by a non-ionic surfactant as disclosed herein and one or more of the hydrophilic therapeutic agents as also disclosed herein (the second plurality of micelles). Like the first plurality of micelles, the hydrophilic therapeutic agent to the non-ionic surfactant may be at a suitable weight ratio to result in suitable particle sizes and/or PDI values. For example, the weight ratio between the hydrophilic therapeutic agent and the non-ionic surfactant may range from about 1:5-1:500, e.g., between about 1:5-1:8, between about 1:5-1:10, between about 1:5-1:20, between about 1:5-1:40, between about 1:5-1:100, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, or between 1:150-1:500.

In some embodiments, the first plurality of micelles, the second plurality of micelles, or both, may have a particle size (diameter) less than 50 nm. In some examples, the particle size may range from about 1 nm to about 50 nm. In some examples, the particle size may range from about 10 nm to about 25 nm. Unless otherwise indicated, the particle size disclosed herein refers to the actual sizes (diameters) of the micelles in a micelle population. For example, if the smallest micelle in a population has a particle size of 10 nm and the largest micelle in the population has a particle size of 50 nm, then the particle size of the population ranges from 10 nm to 50 nm.

In some embodiments, at least 50% (e.g., 60%, 70%, 80%, 90%, or above) of the particles in the first plurality of micelles, the second plurality of micelles, or both, have a particle size (diameter) less than 50 nm.

Alternatively, or in addition, the polydispersity index of the first plurality of micelles, the second plurality of micelles, or both may be less than 0.6, e.g., less than 0.5, less than 0.4, less than 0.3, less than 0.2 or less than 0.1. In some examples, the polydispersity index of the first plurality of micelles, the second plurality of micelles, or both may range from 0 to 0.6, e.g., 0.1 to 0.6, 0.2 to 0.6, 0.3 to 0.6, 0.4 to 0.6, 0.5 to 0.6, 0.1 to 0.5, 0.2 to 0.5, 0.3 to 0.5, 0.4 to 0.5, 0.1 to 0.4, 0.2 to 0.4, 0.3 to 0.4, 0.1 to 0.3, 0.2 to 0.3, or 0.1 to 0.2.

Any of the micelles disclosed herein can be prepared by conventional methods or as disclosed herein. One example is provided below. A suitable amount of an active agent or a hydrophilic therapeutic agent may be mixed with a suitable solvent (e.g., alcohols (e.g., methanol, ethanol, propanol), acetonitrile, chlorinated solvents (e.g., dichloromethane, chloroform) diethyl ether, and ethyl acetate) and the mixture can be stirred (e.g., at 150-500 rpm) at a suitable temperature until the active agent or the hydrophilic therapeutic agent as disclosed herein is full dissolved in the solvent to form a solution. A suitable amount of a pharmaceutically acceptable non-ionic surfactant (e.g., those disclosed herein) can be added to the solution. The resultant mixture can be stirred (e.g., at 100-300 rpm) under a suitable temperature to volatilize the solvent. Once the solvent is completely volatilized, a suitable amount of a pharmaceutically acceptable aqueous solution (e.g., normal saline) can be added to the mixture to produce micelles having the active agent or hydrophilic agent encapsulated. The resultant particles can be filtered through a suitable filter (e.g., a 0.2 μm filter) and the filtered solution comprising drug-containing micelles can be stored in the dark in a refrigerator for future use.

Additional details on the preparation of the micelles of the present technology can be found in U.S. Pat. No. 10,610,496, the relevant disclosures of which are incorporated by reference herein for the subject matter and purpose referenced herein.

### (E) Pharmaceutical Compositions

Any of the active agent, optionally the hydrophilic therapeutic agents, and the non-ionic surfactant, which may form micelles with the active agent and optionally the hydrophilic therapeutic agents, may be formulated into pharmaceutical compositions for use in the therapeutic applications disclosed herein.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes.

Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation. For example, such pharmaceutical compositions may be formulated in a manner suitable for administration via a suitable route, for example, orally, parenterally, topically, rectally, buccally, vaginally or via an implanted reservoir.

A sterile injectable composition, e.g., a sterile injectable aqueous or oleaginous suspension, can be formulated according to techniques known in the art using suitable dispersing or wetting agents (such as Polysorbate 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the compositions described herein may be formulated as a topical formulation, for example, a cream, lotion, or gel for topical application. Such cream, lotion, or gel may be formulated using ingredients known in the art to be appropriate for topical medications.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form more soluble complexes with the oxadiazole compounds, or more solubilizing agents, can be utilized as pharmaceutical carriers for delivery of the oxadiazole compounds. Examples of other carriers include colloidal silicon dioxide, magnesium stearate, sodium lauryl sulfate, and D&C Yellow #10.

In some embodiments, the pharmaceutical composition disclosed herein may further comprise an antioxidant. Examples include, but are not limited to, beta-carotene, lutein, lycopene, bilirubin, vitamin A, vitamin C (ascorbic acid), vitamin E, uric acid, nitric oxide, nitroxide, pyruvate, catalase, superoxide dismutase, glutathione peroxidases, N-acetyl cysteine, and naringenin, or a combination thereof.

### II. Methods for Alleviating Conditions Caused by Abnormal Subcutaneous Deposit of Adipose Tissues and/or Fat

In some aspects, provided herein are methods for alleviating (e.g., treating) conditions caused by abnormal subcutaneous deposit of adipose tissues and/or fat using any of the pharmaceutical compositions disclosed herein.

In some embodiments, conditions caused by abnormal subcutaneous deposit of adipose tissues and/or fat can be tumors of fat tissues, which can result in abnormal deposit of fat cells/tissues (adipose tissues) and/or fat under a skin spot (subcutaneous). In some examples, the tumor of fat tissues can be benign, for example, lipoma. In other examples, the tumor of fat tissues can be malignant, for example, liposarcoma.

### (A). Lipoma and Lipoma-Associated Diseases

Lipomas are benign tumors of fat, formed between the skin and the underlying muscle. Lipomas can grow anywhere in the body where fat cells are present. Neck, shoulders, armpits, and thigh are common places where lipoma would develop. Depending on their location, lipomas can be classified into three subtypes: subcutaneous, intermuscular, and visceral lipomas.

Lipomas can be associated with various medical conditions, for example, Dercum's disease (Adiposis dolorosa), Gardner syndrome, Familial multiple lipomatosis (Hereditary multiple lipomatosis), Madelung's disease (multiple symmetric lipomatosis), painful lipomas, angiolipomas, lipomatosis, subcutaneous adipose tissue diseases, lipedema, and rare adipose disorders (RADs), Panniculitis, Weber-Christian disease, Progressive lipodystrophy or other diseases/symptoms related to fatty lesions. Some of such disorders may result in severe symptoms such as swelling, serious pain or even mental issues.

In some embodiments, a subject suitable for the treatment disclosed herein may be a human patient having at least one lipoma. Such a human patient may also have a fat disorder, subcutaneous adipose tissue disease, lipedema, a rare adipose disorder (RADs), skin disease, a lipid metabolism disorder, or a metabolic disease. Alleviating lipoma by any of the methods disclosed herein is expected to benefit treatment of such diseases and disorders.

In some examples, provided herein is a method for treating Dercum's disease (DD), using the any of the compositions disclosed herein. DD, also known as Adiposis dolorosa, lipomatosis dolorosa, or Ander's syndrome, is a rare disorder characterized by chronic pain due to multiple lipomas in the adipose tissue. The disease is often accompanied by a number of associated symptoms such as weakness, fatigue, depression, and dementia. The painful adipose tissue can be diffuse and generalized or concentrated in palpable lipomas. The most commonly affected locations are the extremities, the trunk, the pelvic area, and the buttocks. DD patients often can be diagnosed by exclusion and imaging is helpful in DD diagnosis. Patients with Dercum's disease typically suffer from severe pain in their fatty tissue deposits, which often become more intense during the course of the disease. Such pain is particularly frequent in the abdomen and buttocks, the upper arm and lateral thighs, and sometimes also in the knees and elbows. Even minimal irritation (e.g., light contact, draughts, rubbing of clothes or bed linen, temperature change) can cause severe discomfort. This can also lead to psychosocial problems. The diagnosis of Dercum's disease should be based on clinical examination and after excluding relevant differential diagnoses. Two main criteria were formulated in a 2012 publication (Hansson E, Svensson H, Brorson H. Review of Dercum's disease and proposal of diagnostic criteria, diagnostic methods, classification and management.): obesity and chronic pain (>3 months) in hyperplastic/hypertrophic adipose tissue. There is no unambiguous histological or instrumental (imaging) diagnostic criteria.

Table 1 below summarizes differential diagnoses and co-morbidity of DD. Table 2 lists four subtypes based on its morphological appearance and distribution pattern.

TABLE 1
Differential diagnoses and comorbidity of Dercum's disease
Differential diagnoses and comorbidity of Dercum's disease

Fibromyalgia
Lipedema, lipolymphedema, elephantiasis
Cushing's disease, Cushing syndrome
Multiple symmetric lipomas
Neurofibromatosis type I
Panniculitis (traumatic, autoimmune, infectious)
(Deep) erythema nodosum, nodular erythemas
Somatized depression (with obesity)
Medication, drug effect or withdrawal
Peripheral neuropathies
Mucinoses
Granulomatous disorders (infectious or auto-inflammatory)

TABLE 2
Classification of Dercum's disease
Symptoms

| | | |
|---|---|---|
| Type I | Generalized | Diffusely distributed, painful |
| | diffuse type | fatty tissue without clearly |
| | | demarcated lipomas |
| Type II | Generalized | Generalized pain in fatty tissue |
| | nodular type | and intensive pain in multiple |
| | | lipomas |
| Type III | Localized | Pain within and limited to |
| | nodular type | multiple lipomas |
| Type IV | Juxta-articular | Solitary fatty tissue tumors on |
| | type | medial knees or elbows |

Effective treatment for patients with Dercum's disease is currently lacking as there is no effective long-term treatment. Management of Dercum's disease typically involves pain control through oral medications or direct injections or surgical removal. Surgical interventions (e.g., liposuction and lipectomy) have varying outcomes. It has been proven that direct injection of lidocaine into lipomas can only provide short-term pain relief.

### (B). Liposarcoma and Liposarcoma-Associated Diseases

Liposarcoma is a rare type of malignant tumor that develops in fatty tissues. It is a type of soft tissue sarcoma. Liposarcoma can begin in the fat cells anywhere in the body. Typically liposarcoma occurs in older adults; however, it can happen in any age group. Symptoms of liposarcoma depend on the part of the body where the malignant tumor forms. For example, liposarcoma in the arms and legs may cause one or more of the following symptoms: a growing lump of tissue under the skin, pain, swelling, and weakness of the affected limb. Liposarcoma in the belly can cause abdominal pain, abdominal swelling, feeling full sooner when eating constipation, and/or blood in stool. Current treatment for liposarcoma usually involves surgery to remove the malignant tumor. Other treatments such as radio therapy may also be used.

Liposarcoma can be associated with various medical conditions, for example, Soft tissue sarcoma, Lipomatous hemangiopericytoma.

In some examples, provided herein is a method for treating liposarcoma, using the any of the compositions disclosed herein. Liposarcoma is classified as a malignant tumor because of its potential to recur locally and spread to other areas of the body. The severity of disease depends on the subtype of the liposarcoma and the presenting stage of the primary tumor. It can arise in various locations throughout the body, although it is most frequently found in the extremities particularly in the thigh. It can also grow in the back of the abdomen in an area called the “retroperitoneum” where, because of the vast amount of space, can effectively hide a tumor of substantial size and weight. Some individuals with liposarcoma may not have symptoms in the early stages, but as the tumor grows and advances to later stages, it can potentially compress other tissues and cause pain.

The specific genetic cause of liposarcoma has yet to be identified, though studies have proposed that it starts in fat cells that have lost their ability to mature or have unregulated growth. It has been found to be more common in middle-aged males from 50-65 years of age compared to females and is very rare in children.

Most patients who are diagnosed with liposarcoma do not have any early symptoms and it will unnoticed during the initial stages of the disease until the tumor has grown to a large enough size to compress neighboring tissues and cause pain or decreased function. Sometimes, it be noticed as a deep-seated mass to touch. Liposarcoma, as with all other malignant tumors, can present with non-specific symptoms such as fevers, chills, fatigue, night sweats and weight loss. If the tumor is retroperitoneal in location, it can present with specific symptoms in the abdomen, including abdominal or flank pain, swelling, and constipation or the sensation of feeling full sooner than expected after eating.

The most critical step in the diagnosis of liposarcoma involves taking a biopsy of the mass of concern. A biopsy is when tissue is retrieved from the tumor in order to be evaluated under the microscope to assess whether or not the tissue has tumor-specific features. Since many of these tumors are deeply embedded into the body, imaging such as ultrasound can be used to guide where the needle is relative to the mass and ensure that the tissue sample is retrieved specifically from that mass.

Liposarcoma can also be diagnosed by imaging the body either by computed tomography (CT) or magnetic resonance imaging (MRI). CT uses multiple X-ray measurements to create an image of the body and it is important in assessing the location of a mass and its relationship to surrounding tissues. MRI is another way to image liposarcoma, and it can show characteristics of the mass itself which might be helpful in diagnostic differences between benign and malignant soft tissue masses.

TABLE 3
Classification of Liposarcoma
Subdivisions

| | |
|---|---|
| Type I | well differentiated (adipocytic, sclerosing, and inflammatory) |
| Type II | dedifferentiated |
| Type III | myxoid |
| Type IV | round cell |
| Type V | pleomorphic |

Table 3 lists five subtypes of liposarcoma: well differentiated, dedifferentiated, myxoid, round cell and pleomorphic. The well differentiated type is less aggressive and tends to be a large painless mass found in deeper tissues and in the retroperitoneum. Myxoid, round cell and pleomorphic types tend to be in the arms and legs, whereas dedifferentiated tends to be in the retroperitoneum and often associated with the well differentiated variety. Specifically, pleomorphic liposarcoma is the least common subtype with a high rate of recurrence and poor outcomes.

### (C). Treatment of Conditions Caused by Abnormal Subcutaneous Deposit of Adipose Tissues and/or Fat

To practice the method disclosed herein, an effective amount of the pharmaceutical composition described herein can be administered to a subject (e.g., a human) in need of the treatment via a suitable route, such as parental administration or local administration (e.g., local injection or topical administration. Subjects (e.g., humans) that benefit from such an administration include patients having a lipoma or liposarcoma or suspected of having a lipoma or liposarcoma. In some examples, the method disclosed herein aims at treating Dercum's disease in human patients. In other examples, the method disclosed herein aims at treating liposarcoma.

Among those with Dercum's disease, four subgroups of patient populations are particularly amenable to the described treatment and are described below.

Subgroup type I refers to a generalized diffuse type of lipoma. Lipomas of this subgroup may not be clearly demarcated but are diffusely distributed in fatty tissue. The lipoma is frequently associated with a pain response.

Subgroup II is a generalized nodular type of lipoma. The nodular lipomas reside generally in fatty tissue and is generally associated with a pain response in the fatty tissue and in the lipoma. Additionally, this type of subgroup can lead to multiple lipomas.

Subgroup III is a localized nodular type of lipoma. This subtype generally leads to multiple lipomas, which are painful specifically in the lipoma.

Subgroup IV is a juxta-articular type of lipoma. This type of lipoma is frequently a fatty tissue tumor on medial knees or elbows.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result. In some embodiments, the desired result is the reduction or elimination of a lipoma or liposarcoma, or alleviation of at least one symptom associated with a lipoma-associated disease (e.g., pain) or a liposarcoma-associated disease. Methods of determining whether a dosage resulted in the desired result would be evident to one of skill in the art. The particular dosage regimen, i.e., dose, timing and repetition, used in the method described herein will depend on the particular subject and that subject's medical history. Treatment efficacy for a target disease/disorder can be assessed by methods well-known in the art.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, by topically or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

In one embodiment, a pharmaceutical composition disclosed herein is administered via site-specific or targeted local delivery techniques. Examples of site-specific or targeted local delivery techniques include various implantable depot sources of the composition or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

In some examples, the pharmaceutical composition disclosed herein is administered to a subject by injection, for example, intramuscular (IM) injection, subcutaneous (SC) injection, intravenous (IV) injection, intraosseous injection, epidural injection, intradermal (ID) injection, or any other injected forms.

In some instances, the pharmaceutical composition is injected to the subject on or near a lipoma site. In other examples, the pharmaceutical composition is for topical administration.

In some instances, a pharmaceutical composition as disclosed herein is administered to a subject via injection at the lipoma site or liposarcoma site (at or near the lipoma site or liposarcoma site) at an amount (a therapeutically effective amount) sufficient to reduce the size of the lipoma or liposarcoma. The injection volume of the pharmaceutical composition may be determined according to the diameter of the lipoma or liposarcoma. For example, the therapeutically effective amount of the pharmaceutical composition may contain about 1 to 25 mg (e.g., about 1 to 5 mg, about 5 to 10 mg, about 10 to 15 mg, about 15 to 20 mg, or about 20 to 25 mg) of the active agent per injection to each extra-small size lipoma, which refers to lipomas having a diameter about 1.0 to 2.0 cm. In another example, the therapeutically effective amount of the pharmaceutical composition may contain about 2.5 to 60 mg (e.g., about 2.5 to 10 mg, about 10 to 20 mg, about 20 to 30 mg, about 30 to 40 mg, about 40 to 50 mg, or about 50 to 60 mg) of the active agent per injection to each small size lipoma or liposarcoma, which refers to lipomas or liposarcomas having a diameter about 2.0 to 3.5 cm. In yet another example, the therapeutically effective amount of the pharmaceutical composition may contain about 10 to 200 mg (e.g., about 10 to 50 mg, about 50 to 100 mg, about 100 to 150 mg, or about 150 to 200 mg) of the active agent per injection to each medium size lipoma or liposarcoma, which refers to lipomas or liposarcomas having a diameter about 3.5 to 5.0 cm. In other examples, the therapeutically effective amount of the pharmaceutical composition may contain about 30 to 300 mg of the active agent per injection to each large size lipoma or liposarcoma, which refers to lipomas or liposarcomas having a diameter greater than 5 cm (e.g., about 5-6 cm). In other examples, the therapeutically effective amount of the pharmaceutical composition may contain about at least 45 mg of the active agent per injection to each extra-large size lipoma or liposarcoma, which refers to lipomas or liposarcomas having a diameter greater than 6 cm.

Any of the pharmaceutical compositions may be administered to the subject at least once, depending on the size of the lipoma or liposarcoma for treatment.

In one embodiment, the pharmaceutical composition is administered to the subject by topical administration. In some instances, the therapeutically effective amount of the composition may contain about 0.5 to 5 mg/cm² (e.g., about 0.5 to 1 mg/cm², about 1 to 2 mg/cm², about 2 to 3 mg/cm², about 3 to 4 mg/cm², or about 4 to 5 mg/cm²) of the active agent.

Any of the pharmaceutical compositions may be administered to a subject as disclosed herein at an amount sufficient to achieve one or more of the following results, measured by lipomas sites analysis: (a) reducing the diameter of the lipoma by at least 2 mm or 5%; (b) decrease the height of the lipoma by at least 2 mm or 5%; (c) reduce the overall volume of the lipoma by at least 10%; and (d) reduce the surface area of the lipoma by at least 5%.

Any of the pharmaceutical compositions may be administered to a subject as disclosed herein at an amount sufficient to achieve one or more of the following results, measured by liposarcoma sites analysis: (a) reducing the diameter of the liposarcoma by at least 2 mm or 5%; (b) decrease the height of the liposarcoma by at least 2 mm or 5%; (c) reduce the overall volume of the liposarcoma by at least 10%; and (d) reduce the surface area of the liposarcoma by at least 5%.

Alternatively or in addition, any of the pharmaceutical compositions may be administered to a subject in need of the treatment via a suitable administration route at an amount sufficient to decreases at least one level pain score according to comparative pain scale.

In some embodiments, any of the pharmaceutical compositions may be administered to a subject in need of the treatment via a suitable administration route at an amount sufficient to improve the total score at least one point according to QOL evaluated by the 36-Item Short Form Survey (SF-36) and the Psychological General Well Being Index (PGWBI).

### III. Kits for Use in Treatment of Conditions Caused by Abnormal Subcutaneous Deposit of Adipose Tissue and/or Fat

The present disclosure also provides kits for use in treating or alleviating conditions caused by abnormal subcutaneous deposit of adipose tissue and/or fat (e.g., lipoma or liposarcoma or a target disease associated with lipoma or liposarcoma, such as those described herein (e.g., DD)). The present disclosure also provides kits for use in treating or alleviating liposarcoma or a target disease associated with liposarcoma, such as those described herein. Such kits can include one or more containers comprising the pharmaceutical composition disclosed herein. In some instances, the pharmaceutical composition may be co-used with a second therapeutic agent.

In some embodiments, the kit can comprise instructions for use in accordance with any of the methods described herein. The included instructions can comprise a description of administration of the pharmaceutical composition, and optionally the second therapeutic agent, to treat, delay the onset, or alleviate lipoma or liposarcoma or a target disease associated with such as those described herein. The kit may further comprise a description of selecting an individual suitable for treatment based on identifying whether that individual has lipoma or liposarcoma or the target disease, e.g., applying the diagnostic method as described herein.

The instructions relating to the use of the pharmaceutical composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readble instructions (e.g., instructions carried on a magnetic or optical storage disk) are also acceptable.

The label or package insert indicates that the composition is used for treating, delaying the onset and/or alleviating lipoma or liposarcoma or an associated disease, such as DD or liposarcoma. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Preparation of Curcumin-Containing Micelles and Characterization Thereof

Exemplary curcumin-containing partial micellar formulations and micellar formulations were prepared as follows.

### (a) Curcumin-ELP Partial Micellar Formulation

20 g of polyoxyl 35 castor oil (Kolliphor® ELP, also known as Cremophor® ELP) was mixed with an appropriate amount of normal saline for injection to a total weight of 100 g. The solution was mixed well to completely dissolve ELP to obtain a 20% ELP solution. 400 mg of curcumin was mixed with an appropriate amount of the 20% ELP solution to a total weight of 80 g. The solution was mixed well to completely dissolve curcumin to obtain a curcumin ELP partial micellar formulation, which has a curcumin concentration of approximately 5 mg/mL, and an ELP concentration of approximately 20% (by weight). The weight ratio of curcumin to ELP was approximately 1:40.

### (b) Curcumin-ELP Micellar Formulation

450 mg of curcumin was mixed with 80-140 mL of dichloromethane and stirred at 150-500 rpm at room temperature until curcumin dissolved completely. 18 g of polyoxyl 35 castor oil (Kolliphor® ELP, also known as Cremophor® ELP) was added to the solution and stirred well at 100-300 rpm to volatilize dichloromethane. Once dichloromethane volatilized completely, normal saline was slowly added to a total volume of 90 mL, and the solution was mixed well to obtain the curcumin-ELP solution, which has a curcumin concentration of 5 mg/mL and an ELP, concentration of approximately 20% (wt %). The weight ratio of curcumin to ELP was 1:40 in the resultant solution.

### (c) Curcumin-HS-15 Partial Micellar Formulation

20 g of polyoxyl 15 hydroxystearate (Kolliphor® HS-15 (HS-15)) was mixed with an appropriate amount of normal saline for injection to a total weight of 100 g. The solution was mixed well to completely dissolve HS-15 to obtain a 20% HS-15 solution. 400 mg of curcumin was mixed with an appropriate amount of the 20% HS-15 solution to a total weight of 80 g. The solution was mixed well to completely dissolve curcumin to obtain the curcumin HS-15 partial micellar formulation, which has a curcumin concentration of approximately 5 mg/mL and an HS-15 concentration of approximately 20% (wt %). The weight ratio of curcumin to HS-15 was approximately 1:40.

### (d) Curcumin-HS-15 Micellar Formulation

500 mg of curcumin was mixed with 80-140 mL of dichloromethane and stirred at 150-500 rpm at room temperature until curcumin dissolved completely. 20 g of polyoxyl 15 hydroxystearate (Kolliphor® HS-15 (HS-15)) was added and stirred at 100-300 rpm to volatilize dichloromethane. Once dichloromethane volatilized completely, normal saline was slowly added to a total volume of 100 g. The solution was mixed well to form drug-containing micelles to obtain the curcumin HS-15 micellar formulation, which has a curcumin concentration of approximately 5 mg/mL and an HS-15 concentration of approximately 20% (by weight). The weight ratio of curcumin to HS-15 was approximately 1:40.

The curcumin-ELP partial micellar formulation (a), curcumin-HS-15 partial micellar formulation (c), curcumin-ELP micellar formulation (b), and curcumin-HS-15 micellar formulation (d) were analyzed by a particle size analyzer to determine presence of micelles and if so, size of the particles. The results showed that both the curcumin-ELP partial micellar formulation and curcumin-HS-15 partial micellar formulation showed curcumin precipitates, and had a lower number of curcumin-containing micelles. On the contrary, the curcumin-ELP micellar formulation and curcumin-HS-15 micellar formulation were clear without any stratification (indicating no curcumin precipitation) and had a higher number of curcumin-containing micelles.

In addition, the particle sizes (indicated by mean diameters) of the micelles in the curcumin-ELP partial micellar formulation, curcumin-HS-15 partial micellar formulation, curcumin-ELP micellar formulation, and curcumin-HS-15 micellar formulation were 13.16±0.18 nm, 13.18±1.45 nm, 12.43±0.40 nm, and 11.46±0.41 nm, respectively, and the PDI values were 0.22±0.03, 0.18±0.05, 0.28±0.05, and 0.18±0.04, respectively.

More details can be found in US 10,610,496, the relevant disclosures of which are incorporated by reference for the subject matter and purpose referenced herein.

### Example 2: Method for Treating Dercum's Disease (DD)

Dercum's disease (DD) is a chronic and recurrent severe rare disease. DD patients typically have multiple lipomas or fat masses with abnormal accumulation of subcutaneous fat on the trunk and limbs, which can be accompanied with spontaneous severe pain in the affected area and can last for more than 3 months. The severe chronic pain often makes the patient unable to live a normal life.

The symptoms of DD usually would not spontaneously disappear and the lipomas or fat masses may become larger in a short period.

It is estimated that there are currently about 124,500 Dercum's disease patients in the United States, and about 240,000 DD patients in Europe. The occurrence rate of DD in women is about 5 to 30 times more than in men. At present, there is no drug or product approved for marketing in the world to cure Dercum's disease. Current treatment approaches for DD include surgery and drugs for relieving symptoms. Liposuction or surgical excision are relatively common treatment methods, but they are associated with side effects and high risks and require a long postoperative recovery period. Even after surgical removal or liposuction, new lipomas or fat masses would grow again in most cases.

This example illustrates an exemplary clinical trial to evaluate safety and efficacy of a curcumin-containing composition as an example in human patients (including both male and female patients) having nodular Dercum's disease (DD).

Eligible participants have at least 4 painful and well-defined lipomas of appropriate size, which is to be selected as the treatment area. Each enrolled participant receives one or more courses of the treatment with their allocated dose injected to the treatment area.

The injection volume per lipoma is based on the lipoma size (as determined by ultrasound). The total dose on multiple lipomas in each treatment is based on the lipomas size and number of lipomas. The dosing scheme is presented in Table 4 below.

TABLE 4
Dosing Scheme

| | | | | Total |
|---|---|---|---|---|
| | Volume per | | | Injection |
| | Injection | Lipoma | Lipoma | Volume per |
| Example | (mL) | Size | Diameter (cm) | Lipoma (mL) |
| 1 | 2 | Extra- | ≥1.0 and ≤2.0 | 2 |
| | | small | | |
| | | Small | >2.0 and ≤3.5 | 4 |
| | | Medium | >3.5 and ≤5.0 | 6 |
| | | Large | >5.0 and ≤6.0 | 10 |
| 2 | 3 | Extra- | ≥1.0 and ≤2.0 | 3 |
| | | small | | |
| | | Small | >2.0 and ≤3.5 | 6 |
| | | Medium | >3.5 and ≤5.0 | 9 |
| | | Large | >5.0 and ≤6.0 | 15 |

Notes:
The concentration of the composition comprises 4 mg/mL curcumin.

The subject should lie down during the administration. The composition can be administered to the subjects via any size of needle and syringe depending on the operational requirements. After confirming the injection areas, a marker is used to markup the injection spots. The needle should be inserted beside the injection marker to avoid the ink being transferred into the tissue. The needle is inserted into adipose tissue of each identified lipoma to administer the composition evenly. After completing the administration for one lipoma, pressure is applied to each injection site for 10-20 seconds if bleeding is observed to minimize the condition. Right after the injections are completed, the injection sites are messaged with moisturizer or topical antibiotic cream, especially the edge of the injection area to help spread the drug evenly. Make sure no bleeding is around the injection area before rubbing. If it is bleeding, use wound dressing to cover the bleeding site and apply pressure to stop the bleeding. Use the heel of the hand to massage each injection site for approximately 60 seconds. Tiny lumps may appear due to the injection solution under thin skin. Rub the lump until the drug spreads evenly. Subjects are reminded not to massage themselves after discharge as continuous massage may cause inflammation-related AEs.

### Results

Five patients (A, B, C, D and E) have been treated following the treatment conditions noted above. Each patient was given up to two injections. No additional injection was given to a patient when his or her lipoma disappears after the first injection.

Lipoma sizes and pain scales in each of the five patients were examined at the following stages:
V2 (B L): baseline, before treatment
V3: Second treatment (If lipoma disappears after the first treatment, the second treatment is not performed)
V4: 4 weeks after the last treatment
V5: 8 weeks after the last treatment

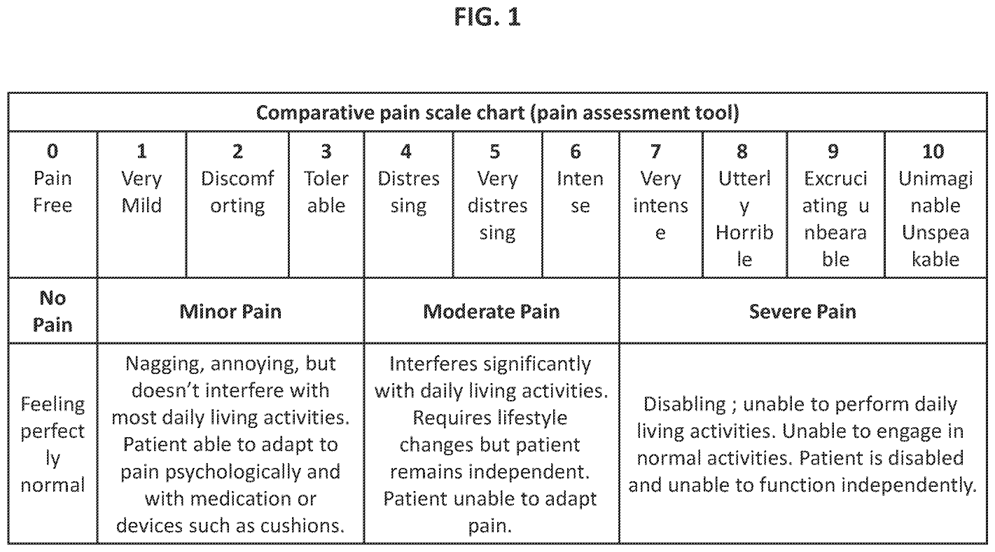

The size of lipoma is measured by ultrasound and the volume is calculated, and the pain is evaluated by the Comparative Pain Scale (0-10 points, the higher the score, the more severe the pain), which is illustrated in FIG. 1 below. See also the Comparative Pain Scale Chart provided at 123rf.com/photo_61431492_stock-vector-faces-pain-scale-doctors-pain-assessment-scale-comparative-pain-scale-chart-faces-pain-rating-tool-v.html, cited August 2018, the relevant disclosures of which are incorporated by reference for the subject matter and purpose referenced herein. The treatment dosage and number of lipomas of each patient is provided in Table 5 below:

TABLE 5
Participant Dosage and Number of Lipomas

| Participant No. | C | E | A | D | B |
|---|---|---|---|---|---|
| Dose | low | low | high | high | high |
| Volume per | 2 | 2 | 3 | 3 | 3 |
| Injection (mL) | | | | | |
| Number of | 4 | 8 | 5 | 8 | 6 |
| lipomas | | | | | |

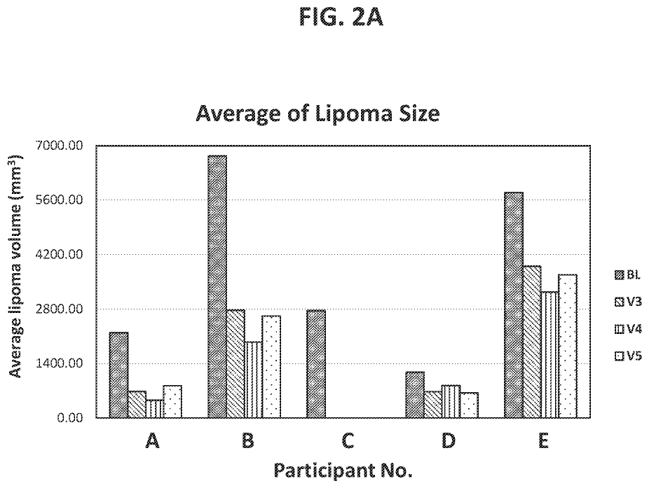

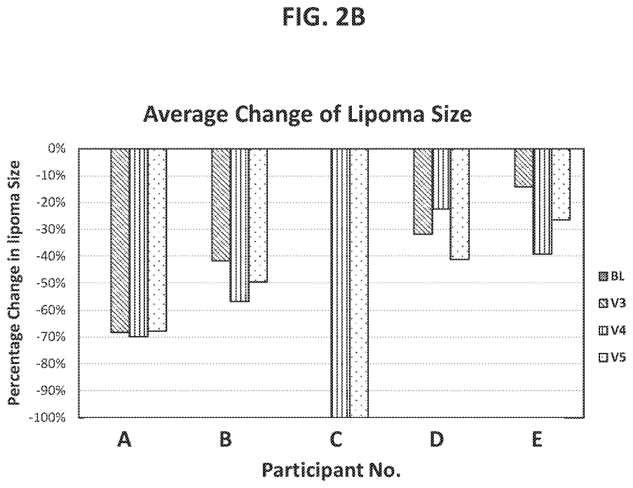

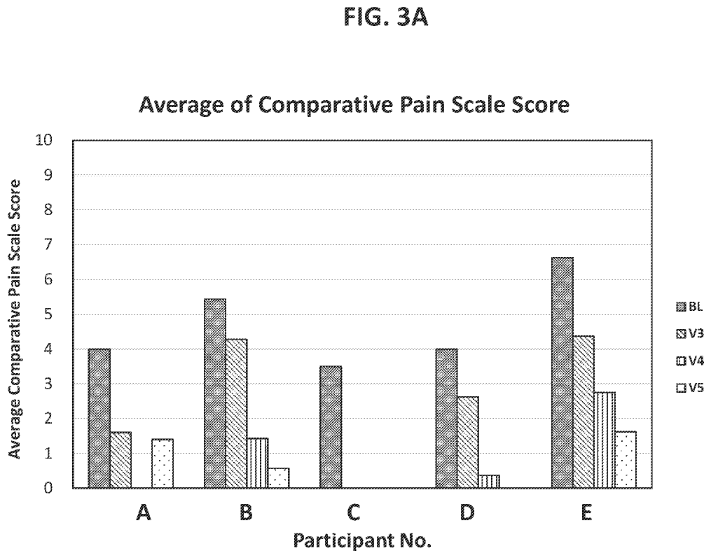

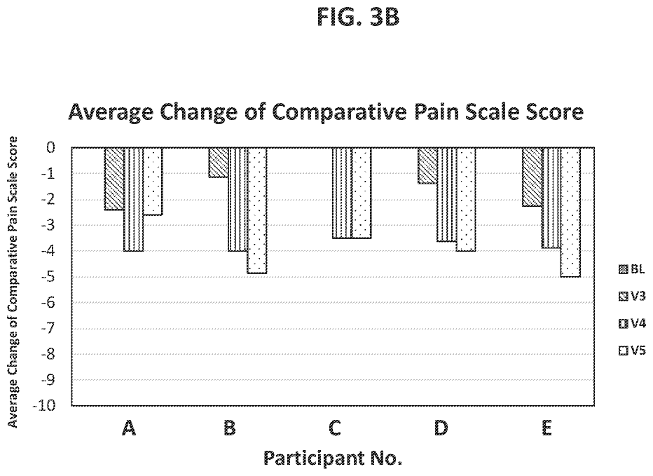

The average lipoma sizes and average changes of lipomas in each patient before and after the treatment are shown in FIG. 2A and FIG. 2B, respectively. The average comparative pain scale scores and average changes of comparative pain scale scores of each patient before and after the treatment are shown in FIG. 3A and FIG. 3B, respectively.

The results obtained from this study indicate that the average lipoma sizes of all patients have been reduced significantly after the treatment. As one example, the lipoma in one patient (patient C) disappeared after the injection treatment.

Further, all patients experienced a significant decrease (e.g., elimination) in Comparative Pain Scale Score after the treatment.

In sum, the results achieved from the clinical study show that the composition used herein, comprising curcumin, is effective in reducing the size of or eliminating lipomas and alleviating pain scores in DD patients. The results also suggest that the compositions disclosed herein would benefit treatment of lipoma and associated diseases, as well as other conditions that are also caused by abnormal subcutaneous deposit of adipose tissues or fat, for example, liposarcoma and associated diseases.

### Example 3: Treatment of Liposarcoma

SW872 is a human malignant liposarcoma cell line isolated from the connective tissue of a male with liposarcoma. The histopathology evaluation reported an undifferentiated malignant tumor consistent with liposarcoma.

Liposarcoma is a rare and heterogeneous soft tissue malignant tumor and has a significant impact on mortality with a poor prognosis. Liposarcoma is the second-highest soft tissue aggressive malignant tumor, with at least 30 cases per million annually in the United States.

Notably, there are still limitations in diagnosing patients with soft tissue liposarcoma due to malignancy of cell types, tumor size, and anatomical location. Currently, the options for treating of liposarcoma patients are chemotherapy, radiotherapy, and surgery, but a favorable response to conventional treatment in liposarcoma remains unsatisfactory. Therefore, there is still a need for novel, non-toxic, and more effective therapeutic agents to comply with the necessity of liposarcoma patients. To date, there is no effective treatment for liposarcoma, whereas surgical resection is only the gold treatment with numerous adverse effects.

TABLE 6
Experimental group

| Group | | Curcumin | | Drug action |
|---|---|---|---|---|
| number | Group | concentration | | duration |
| 1 | Vehicle | — | | 48 hours |
| 2 | DDTA-1 | 0.4 | ppm | |
| 3 | DDTA-2 | 0.8 | ppm | |
| 4 | DDTA-3 | 1.6 | ppm | |
| 5 | DDTA-4 | 3.2 | ppm | |
| 6 | DDTA-5 | 6.4 | ppm | |
| 7 | DDTA-6 | 12.8 | ppm | |
| 8 | DDTA-7 | 25.6 | ppm | |

### SW872 Tumor Cell Toxicity Assay (MTT Assay)

SW872 cells were inoculated at a quantity of 1×10⁵ cells/well. After the cells were full on the next day, different concentrations of test substances were added to act for 48 hours, and then the MTT medium was replaced, and the effect was carried out in a 37° C. incubator for 2 hours.

After the supernatant was removed, DMSO was added to redissolve the purple crystals, and the maximum absorbance value O.D.570 nm and the background value O.D.630 nm were measured with an ELASA reader. The absorbance value of each group was first deducted from the background value, and the data was expressed as [100%-(A_{570 nm}-A_{630 nm})/A_{vehicle}*100%]. This data can be used to compare the cytotoxic effects of different concentrations of test substances on SW872 cells.

### Results

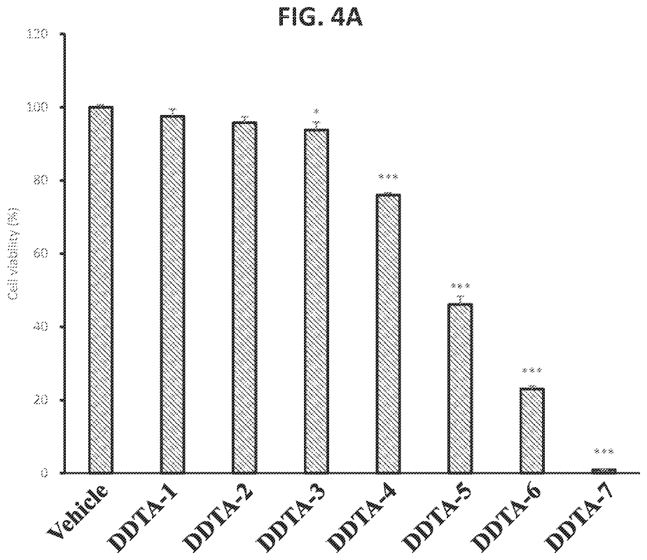

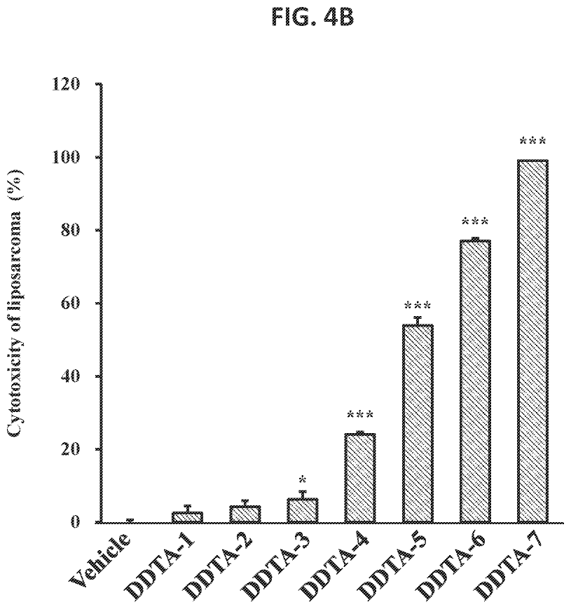

The cell viability (%) and cytotoxicity of liposarcoma (%) in different concentrations of test substances on SW872 cells are shown in FIG. 4A and FIG. 4B, respectively. The test results found that DDTA can significantly reduce the number of SW872 tumor cells starting at 1.6 ppm, 53.9% of tumor cells can be reduced at 6.4 ppm, and more than 99% of tumor cells can be eliminated at 25.6 ppm, achieving an almost complete removal effect. Further analysis results show that DDTA The IC50 for SW872 liposarcoma tumor cells is about 5.6 ppm.

TABLE 7A
Cell Viability (%)

| Group | Vehicle | DDTA-1 | DDTA-2 | DDTA-3 | DDTA-4 | DDTA-5 | DDTA-6 | DDTA-7 |
|---|---|---|---|---|---|---|---|---|
| Mean | 100.0 | 97.5 | 95.7 | 93.8 | 76.0 | 46.1 | 23.0 | 0.9 |
| SD | 0.6 | 1.9 | 1.7 | 2.1 | 0.6 | 2.3 | 0.8 | 0.0 |

TABLE 7B
Cytotoxicity of Liposarcoma (%)

| Group | Vehicle | DDTA-1 | DDTA-2 | DDTA-3 | DDTA-4 | DDTA-5 | DDTA-6 | DDTA-7 |
|---|---|---|---|---|---|---|---|---|
| Mean | 0.0 | 2.5 | 4.3 | 6.2 | 24.0 | 53.9 | 77.0 | 99.1 |
| SD | 0.6 | 1.9 | 1.7 | 2.1 | 0.6 | 2.3 | 0.8 | 0.0 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for alleviating a condition caused by abnormal subcutaneous deposit of adipose tissue or fat in a subject, the method comprising administering to a subject in need thereof an effective amount of a composition comprising a first active agent, which comprises (i) a resveratrol compound, a curcumin compound, a quercetin compound, a puerarin compound, or a combination thereof, and (ii) a pharmaceutically acceptable carrier.

2. The method of claim 1, wherein the resveratrol compound is resveratrol, the curcumin compound is curcumin, the quercetin compound is quercetin, and/or the puerarin compound is puerarin.

3. The method of claim 1, wherein the pharmaceutically acceptable carrier comprises a first pharmaceutically acceptable non-ionic surfactant having a hydrophilic-lipophilic balance value (HLB value) greater than 10.

4. The method of claim 3, wherein the first pharmaceutically acceptable non-ionic surfactant comprises Polyoxyl 40 Stearate, Polyoxyl 20 Cetostearyl Ether, Tween 80, polyoxyl 15 hydroxystearate (solutol HS 15), a polyoxyethylene derivative, a polyoxyethylene castor oil derivative, or a combination thereof.

5. The method of claim 3, wherein the first active agent forms a first plurality of micelles with the first pharmaceutically acceptable non-ionic surfactant.

6. The method of claim 3, wherein the weight ratio of the first active agent to the first pharmaceutically acceptable non-ionic surfactant in the composition is 1:2-1:500.

7. The method of claim 5, wherein the micelles in the first plurality of micelles have a diameter ranging from about 1 nm to about 200 nm and/or a polydispersity index (PDI) value less than 0.4.

8. The method of claim 1, wherein the composition is in a form of non-oral dosage form, optionally wherein the composition is formulated for injection, implantation, or transdermal administration.

9. The method of claim 1, wherein the condition is a tumor of fat tissue.

10. The method of claim 9, wherein the tumor of fat tissue is a benign tumor, which optionally is lipoma.

11. The method of claim 10, wherein the tumor of fat tissue is lipoma, which is a nodular lipoma or a diffuse lipoma.

12. The method of claim 9, wherein the tumor of fat issue is a malignant tumor, which optionally is liposarcoma, Proteus syndrome, PTEN hamartoma tumor syndrome, Gardner syndrome, Familial multiple lipomatosis, Cowden syndrome, Madelung's disease, painful lipomas, angiolipomas, lipomatosis, subcutaneous adipose tissue diseases, lipedema, a rare adipose disorder (RAD), or soft tissue sarcoma.

13. The method of claim 1, wherein the condition is Dercum's disease or Lipedema.

14. The method of claim 1, wherein the subject is a human patient having or suspected of having the condition.

15. The method of claim 1, wherein the composition is administered to the subject by a parenteral route.

16. The method of claim 1, wherein the composition is administered to the subject at a local site.

17. The method of claim 16, wherein the composition is administered to the subject topically or by injection.

18. The method of claim 16, wherein the local site is at or near a site where subcutaneous deposit of adipose tissue or fat occurs.

19. The method of claim 18, wherein the local site is at or near a lipoma, or a liposarcoma.
